(19) United States
(12) Patent Application Publication
(10) Pub. No.: US 2023/0079402 A1
(43) Pub. Date: Mar. 16, 2023

(54) METHOD AND DEVICE FOR ACCURATE DETECTION AND PRESENTATION OF ELECTROCARDIOGRAPH SIGNAL COLLECTED BY WEARABLE DEVICE

(71) Applicant: Jiangyu Kangjian Innovation Medical Technology(Chengdu) Co., Ltd, Chengdu (CN)

(72) Inventors: ZHI-BING XU, Hangzhou (CN); PING-HAO LIU, New Taipei (TW); ZHI-BIN HUANG, Shenzhen (CN); WEI-QIANG YOU, Chengdu (CN)

(21) Appl. No.: 17/715,243

(22) Filed: Apr. 7, 2022

(30) Foreign Application Priority Data
Sep. 14, 2021 (CN) 202111073836.0

(51) Int. Cl. A61B 5/00; A61B 5/339; A61B 5/308; A61B 5/352; A61B 5/366

(52) U.S. Cl. CPC A61B 5/7203; A61B 5/308; A61B 5/339; A61B 5/352; A61B 5/366

(57) ABSTRACT

A device for accurately recording and presenting an electrocardiograph (ECG) signal collected by the non-optimal dry metal electrodes of wearable devices includes a first collecting module, a filtering module, a second collecting module, and a controlling module. The first collecting module collects a first ECG signal which is filtered to obtain a high frequency ECG signal and a low frequency ECG signal. The second collecting module collects the high frequency ECG signal and the low frequency ECG signal. The controlling module performs combination processing on the high frequency ECG signal and the low frequency ECG signal, then outputting an effective and accurate ECG signal. The present disclosure also provides a method for detecting and presenting an accurate electrocardiograph signal obtained by a wearable device.

### TECHNICAL FIELD

The present disclosure relates to the technical field of medical technology, in particular to a method and device for detecting the human electrocardiograph signal.

### BACKGROUND

Electrocardiograph (ECG) indicates and records the heart activity in each cardiac cycle, the pacemaker issues a voltage to excite the atrium and the ventricle successively. With the change of bioelectricity, various forms of potential change patterns are drawn from the body surface through the electrocardiograph. Usually, multiple electrode pieces are used to collect the potential difference of multiple parts of the body, and then the continuous signal is generated through analog to digital (AD) conversion chip. As shown in FIG. 1, a typical ECG signal includes a P wave, a QRS complex, and a T wave. In some precise measurement environments, the ECG signal also includes a U wave.

At present, dry metal electrodes are commonly used in ECG acquisition devices on wearable devices. Compared with electrodes wetted with medical gel, the impedance between them is greater than that of skin. The collected ECG signals contain more serious noise interference. Especially in winter, when the skin is dry, the signal-to-noise ratio of ECG signal may fall below 0.5, which seriously affects the recording and presentation of ECG information.

Therefore, improvement is desired.

### BRIEF DESCRIPTION OF THE DRAWINGS

FIG. 1 is a waveform diagram of ECG signal.

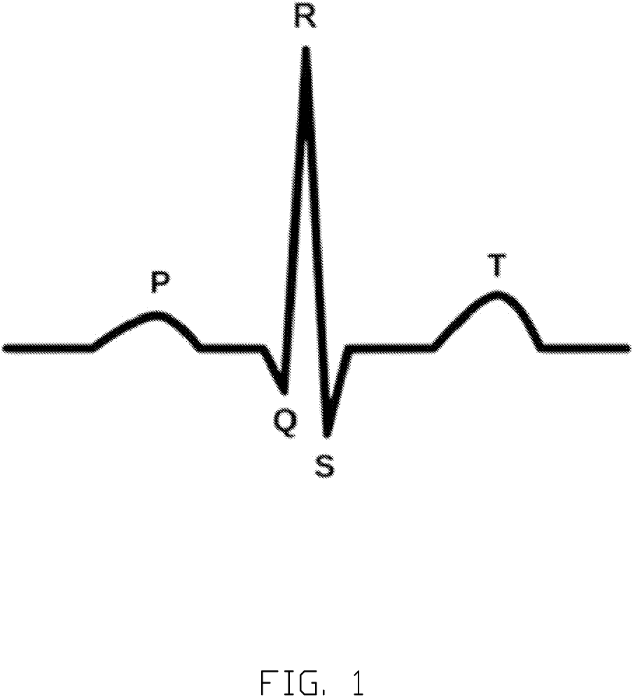

FIG. 2 is a schematic diagram of an electrocardiograph signal detecting device according to an embodiment of the present disclosure.

FIG. 3 is a waveform diagram of the ECG signal of an embodiment of the present disclosure.

FIG. 4 is another waveform diagram of the ECG signal of an embodiment of the present disclosure.

FIG. 5 is a flowchart of a method for detecting electrocardiograph signal according to an embodiment of the present disclosure.

### DETAILED DESCRIPTION

The technical solutions in the embodiments of the present disclosure will be described in conjunction with the accompanying drawings in the embodiments of the present disclosure. Obviously, the described embodiments are part of the embodiments of the present disclosure, and not all of them. Based on the embodiments of the present disclosure, all other embodiments obtained by those of ordinary skill in the art without creative work shall fall within the protection scope of the present disclosure.

The following disclosure provides many different embodiments or examples to implement different structures. In order to simplify the disclosure, the components and settings of specific examples are described below. Of course, they are merely examples and are not intended to limit the present disclosure. In addition, the present application may repeat reference numbers and reference letters in different examples for the purpose of simplification and clarity, which itself does not indicate a relationship between the various embodiments and settings discussed.

Some embodiments of the present disclosure are described in detail below in combination with the accompanying drawings.

The distribution span of band frequencies in electrocardiogram (ECG) signal is large. The frequency band distribution of QRS complex is 15-40 hz, the frequency band distribution of P wave and T wave is 0.8-5 hz, and the frequency of interference signal (such as EMG interference and other Gaussian white noise) is almost full frequency distribution in the whole ECG signal frequency band. The interference signals include power frequency noise, baseline drift, EMG interference, and thermal noise.

The frequency band distribution of the power frequency noise is 50 / 60 Hz. The power frequency noise is generated when collecting the ECG signal, which includes frequency interference and harmonic interference of alternating current (AC) power line. The frequency of the power frequency noise is determined by the municipal power standards adopted in different regions. For example, countries such as China and the European Union adopt 220 V / 50 Hz standard, while countries such as the United States and Japan adopt 110 V / 60 Hz standard. The amplitude distribution of the power frequency noise is 0-0.4 mv, which is equivalent to 5% - 40% of the maximum amplitude of R wave.

The potential on the surface of human body changes due to the activity of muscle fibers, which affects the potential difference measured by the electrode patch on the body surface. The interference caused by this is called EMG interference. The frequency band distribution range of the EMG interference is wide, usually between 0 and 10000 Hz, and more at 30-300 Hz, its frequency characteristic is equivalent to white noise. There is usually a potential of about 30 mV on the surface of human skin, and the amplitude distribution of this signal is 25-35 mv. The EMG interference signal with the maximum amplitude of 5 mv is enough to interfere with the ECG signal.

The thermal noise of electronic components belongs to Gaussian white noise, which is evenly distributed throughout the whole ECG signal frequency band. The thermal noise is caused by the thermal vibration of electrons in conductors, which exists in electronic devices and transmission media.

The EMG interference and the thermal noise of the electronic components will make ECG signal waveform produce small ripples. When collecting the ECG signals, it is generally considered that the frequencies of the EMG interference and the thermal noise of the electronic components are fully distributed throughout the whole ECG signal frequency band.

When the low-pass filter is used to filter the high-frequency noise, it can filter most of the high-frequency EMG interference and thermal noise signals, but at the same time, the high-frequency components in QRS complex are filtered out, and the peak of R wave is cut, which does not meet the standard of medical devices.

One scheme is to filter the baseline drift, power frequency interference and the EMG interference contained in the ECG signal by wavelet decomposition and reconstruction. However, the design process of such scheme is complex, and the calculations are numerous, which makes it difficult to meet the real-time processing requirements of the ECG signals in wearable devices.

Another scheme is to use mean filter and band-pass filter to filter the ECG signals in wearable devices. However, in this scheme, it is difficult to effectively filter the high-frequency EMG interference and the thermal noise, some filtering and denoising methods can better filter the noise distributed in specific frequency band, but the effective signal in the ECG signal coincides with the frequency band distribution of the EMG interference. Effective filtering of the EMG interference in the ECG signal is the difficulty being researched.

The embodiment of the present disclosure provides an electrocardiograph signal detecting device and method. The present disclosure can separate the QRS band with higher frequency from the T-P band with lower frequency in the ECG signal, and different filtering processes can be carried out and then recombined, which simply and effectively filters out high-frequency EMG interference and the thermal noise, while retaining the complete ECG effective signal.

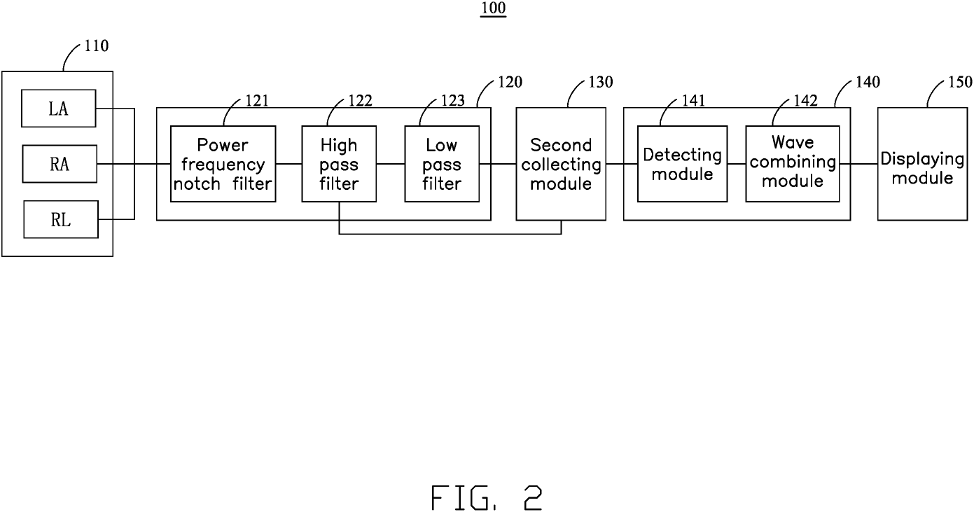

FIG. 2 illustrates an electrocardiograph signal detecting device (electrocardiograph signal detecting device 100) in accordance with an embodiment of the present disclosure.

The electrocardiograph signal detecting device 100 includes a first collecting module 110, a filtering module 120, a second collecting module 130, a controlling module 140, and displaying module 150. The filtering module 120 is electrically connected to the first collecting module 110 and the second collecting module 130. The controlling module 140 is electrically connected to the second collecting module 130 and the displaying module 150. In one embodiment, the first collecting module 110 may be a first collector, and the second collecting module 130 may be a second collector.

The first collecting module 110 is used to collect the first ECG signal. The first ECG signal is a continuous signal generated by analog-to-digital conversion of the potential difference of multiple parts of the body. The first ECG signal includes an ECG effective signal and an interference signal. The interference signals may include power frequency noise signals, baseline drift signals, EMG interference signals, and thermal noise signals.

The first collecting module 110 may include a plurality of dry metal electrodes.

In one embodiment, the first collecting module 110 can include three leads: left arm (LA), right arm (RA) and right leg (RL), each lead can deploy a metal dry electrode.

The filtering module 120 is used to filter the first ECG signal and output a high frequency ECG signal (hereinafter referred to as a third ECG signal) and a low frequency ECG signal (hereinafter referred to as a fourth ECG signal). The third ECG signal and the fourth ECG signal both include the ECG effective signal and part of the interference signal.

In one embodiment, the filtering module 120 may include a power frequency notch filter 121, a high pass filter 122, and a low pass filter 123. The power frequency notch filter 121 is electrically connected to the first collecting module 110 and the high pass filter 122. The low pass filter 123 is electrically connected to the high pass filter 122 and the second collecting module 130. The high pass filter 122 is electrically connected to the second collecting module 130.

The power frequency notch filter 121 is used to filter the power frequency noise signal in the first ECG signal, and output the second ECG signal. The second ECG signal includes the ECG effective signal and part of the interference signal. The center frequency of the power frequency notch filter 121 is 50 / 60 Hz.

The high pass filter 122 is used to filter the baseline drift signal in the second ECG signal and output the third ECG signal. The cut-off frequency of the high pass filter 122 is 0-2 Hz.

The low pass filter 123 is used to filter out the high-frequency noise signal in the T-P band in the third ECG signal and output the fourth ECG signal. The cut-off frequency of the low pass filter 123 is more than 5 Hz.

The second collecting module 130 is used to collect the third ECG signal and the fourth ECG signal.

The controlling module 140 is used to process the third ECG signal and the fourth ECG signal and output the ECG effective signal.

In one embodiment, the controlling module 140 may include a detecting module 141 and a wave combining module 142. The wave combining module 142 is electrically connected to the detecting module 141, the second collecting module 130, and the displaying module 150.

The detecting module 141 is used to detect the R peak of the third ECG signal and identify the complete QRS complex.

The wave combining module 142 is used to perform combination processing on the QRS complex and the fourth ECG signal, and output the ECG effective signal. The combination processing can include calculating filter delay and phase difference, and combining the waveforms of the same frequency band in the QRS complex and the fourth ECG signal to output the ECG effective signal.

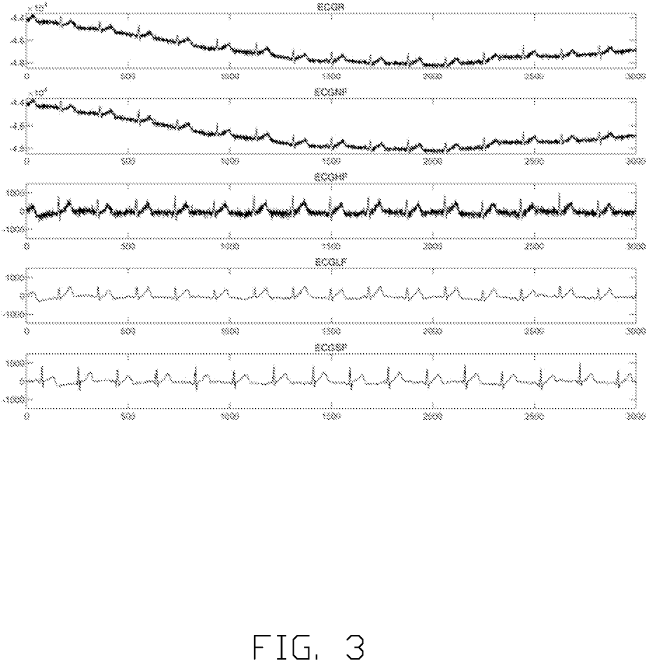

For example, referring to FIG. 3, a signal waveform with a frequency range of 0-3000 Hz can be detected. The ECGR signal is the first ECG signal. The ECGNF signal is the second ECG signal. The ECGHF signal is the third ECG signal. The ECGLF signal is the fourth ECG signal. The ECGSF signal is an effective ECG signal.

The first collecting module 110 collects the ECGR signal through the metal dry electrode. The power frequency notch filter 121 with a center frequency of 50 Hz filters the ECGR signal and outputs the ECGNF signal.

The second order IIR high pass filter with a cut-off frequency of 0.67 hz filters the ECGNF signal and outputs the ECGHF signal. The low pass filter 123 with a cut-off frequency of 10 Hz filters the ECGHF signal and outputs the ECGLF signal. The detecting module 141 detects the R peak of the ECGHF signal and outputs a complete QRS complex. The wave combining module 142 performs processing on the QRS complex and the ECGLF signal and outputs the ECGSF signal.

It can be seen from FIG. 3 that there are a large number of burrs and ripples in the waveform of the ECGHF signal after power frequency notch and high pass filtering, indicating that the ECGHF signal includes the high-frequency EMG interference and the thermal noise signal. After the low-pass filtering, some high-frequency components in the QRS complex in the ECGHF signal are also found to be filtered out. The amplitude of R wave is decreased from 1000 to 500, resulting in the R wave being clipped.

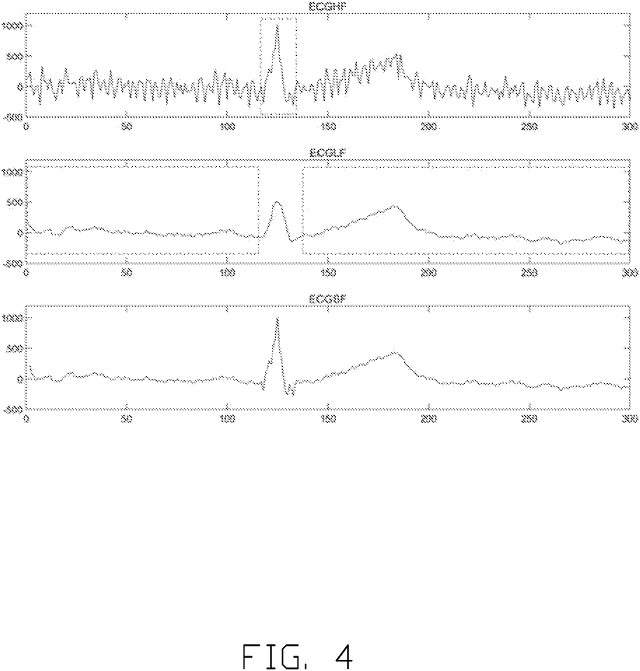

Referring to FIG. 4, the ECGHF signal, the ECGLF signal, and the ECGSF signal in the frequency range of 0-300 Hz are intercepted. The wave combining module 142 obtains the waveforms of the ECGHF signal and the ECGLF signal in the same frequency band by calculating the filter delay and the phase difference, and then obtains the complete waveform of the ECGSF signal through combination processing.

It can be seen from FIG. 4 that the complete QRS band in the ECGHF signal is first identified, and then the high-frequency EMG interference and the thermal noise signal are filtered through low-pass filtering, and combined processing is applied to the T-P band in the ECGLF signal and QRS band in the ECGHF signal. These are recombined into the ECGSF signal in the process, with the high-frequency noise filtered out and the R peak amplitude retained to obtain a complete ECG effective signal.

The controlling module 140 may be a processor. The processor may include one or more processing units. For example, the processor may include, but is not limited to, an application processor (AP), a modulation and demodulation processor, a graphics processing unit (GPU), an image signal processor (ISP), a controller, a video codec, a digital signal processor (DSP), a baseband processor, a neural network processing unit (NPU). It can be integrated in one or more separate processing units.

A storage device can be set in the processor to store instructions and data. In some embodiments, the storage device in the processor is a cache memory. The storage device can store instructions or data just created or recycled by the processor. If the processor needs to use the instruction or data again, it can be called up directly from the storage device.

The displaying module 150 is used to display the ECG effective signals.

The displaying module 150 may be a display screen. The display screen includes a display panel. The display panel can be, but is not limited to, liquid crystal display (LCD), organic light emitting diode (OLED), active-matrix organic light emitting diode or active-matrix organic light emitting diode (AMOLED), flexible light emitting diode (FLED), mini-LED, micro-LED, micro-OLED, quantum dot light emitting diode (QLED). In some embodiments, the electrocardiograph signal detecting device 100 may include one or more (N) display screens, where N is a positive integer greater than 1.

In another embodiment, the electrocardiograph signal detecting device 100 may further include a storage device (not shown in figures). The storage device may include an external storage interface and an internal storage device. The external storage interface can be used to connect an external storage card, such as a micro-SD card, to expand the storage capacity of the electrocardiograph signal detecting device 100. The external storage card communicates with the controlling module 140 through the external storage interface to realize the data storage function. The internal storage device can be used to store computer executable program code, which includes instructions. The internal storage device can be used to store computer executable program code, which includes instructions. The internal storage device may include a program storage area and a data storage area. The storage data area can store data (such as audio data, text data) created during the use of the electrocardiograph signal detecting device 100. In addition, the internal storage device may include high-speed random-access memory and nonvolatile memory, such as at least one disk storage device, flash memory device, universal flash storage (UFS). The controlling module 140 executes various functional applications and data processing of the electrocardiograph signal detecting device 100 by running instructions stored in the internal storage device or instructions stored in the storage device set in the controlling module 140, such as realizing the electrocardiograph signal detecting method of the embodiment of the present disclosure.

It can be understood that the electrocardiograph signal detecting device 100 may be a wearable device. The wearable device may include at least one of accessory types (such as watches, rings, bracelets, anklets, necklaces, glasses, contact lenses or head mounted devices (HMDS)), fabric or clothing integration types (such as electronic clothing), body mounting types (such as skin pads or tattoos), and bio implantable types (such as implantable circuits).

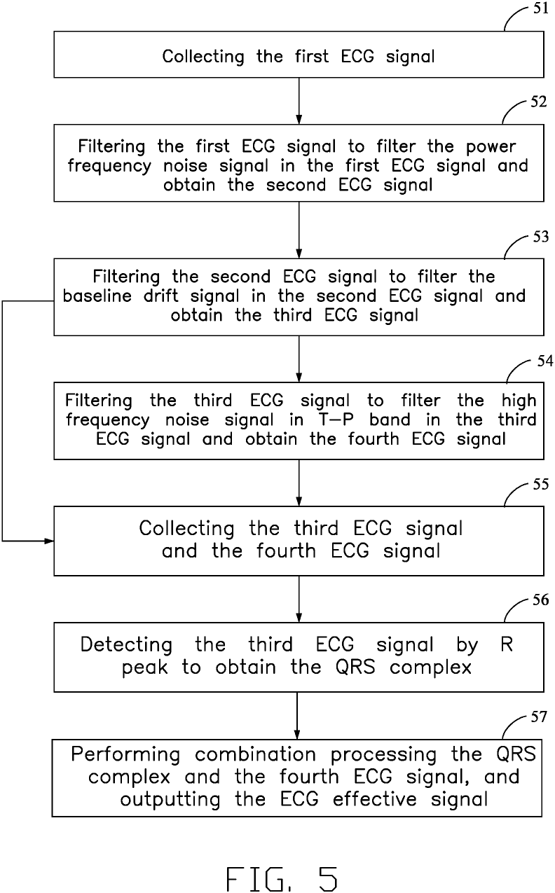

FIG. 5 is a flowchart depicting an embodiment of an electrocardiograph signal detecting method. The electrocardiograph signal detecting method can be applied to the electrocardiograph signal detecting device 100.

Each block shown in FIG. 5 represents one or more processes, methods, or subroutines, carried out in the example method. Furthermore, the illustrated order of blocks is illustrative only and the order of the blocks can change. Additional blocks can be added or fewer blocks may be utilized, without departing from the present disclosure. The example method can begin at block 51.

At block 51, collecting the first ECG signal.

The first ECG signal is a continuous signal generated by analog-to-digital conversion of the potential difference of multiple parts of the body. The first ECG signal includes an ECG effective signal and an interference signal. The interference signals may include power frequency noise signals, baseline drift signals, EMG interference signals, and thermal noise signals.

In one embodiment, the first collecting module 110 can collect the first ECG signal through the metal dry electrodes deployed on the leads LA, RA and RL.

At block 52, filtering the first ECG signal to filter the power frequency noise signal in the first ECG signal and obtain the second ECG signal.

The second ECG signal includes ECG effective signal and part of interference signal. In one embodiment, a power frequency notch filter 121 with a center frequency of 50 / 60 Hz can be used to filter the first ECG signal to obtain the second ECG signal.

At block 53, filtering the second ECG signal to filter the baseline drift signal in the second ECG signal and obtain the third ECG signal.

The third ECG signal includes the ECG effective signal and part of the interference signal.

In one embodiment, a high pass filter 122 with a cut-off frequency of 0-2 Hz may be used to filter the second ECG signal to obtain the third ECG signal.

At block 54, filtering the third ECG signal to filter the high frequency noise signal in T-P band in the third ECG signal and obtain the fourth ECG signal.

The fourth ECG signal includes the ECG effective signal and part of the interference signal.

In one embodiment, a low pass filter 123 with a cut-off frequency of more than 5 Hz can be used to filter the third ECG signal to obtain the fourth ECG signal.

At block 55, collecting the third ECG signal and the fourth ECG signal.

Referring to FIG. 3, the third ECG signal after high pass filtering retains the complete QRS complex. In the fourth ECG signal after low-pass filtering, the high-frequency component of the QRS complex is filtered out and the R wave is peaked.

At block 56, detecting the third ECG signal by R peak to obtain the QRS complex.

In one embodiment, the detecting module 141 can detect the R peak of the third ECG signal and identify the complete QRS complex.

At block 57, performing combination processing the QRS complex and the fourth ECG signal, and outputting the ECG effective signal.

The combination processing may include calculating the filter delay and the phase difference, and combining the waveforms of the same frequency band in the QRS complex and the fourth ECG signal.

In one embodiment, the wave combining module 142 performs combination processing the QRS complex and the fourth ECG signal, and outputs the ECG effective signal.

The embodiment of the present disclosure filters out the power frequency noise and the baseline drift through power frequency notch and high pass filtering to identify the complete QRS band in the third ECG signal. Then, the high-frequency EMG interference and thermal noise are filtered by low-pass filtering. The T-P band in the fourth ECG signal and the QRS band in the third ECG signal are combined to form an effective ECG signal. The embodiment of the present disclosure can simply and effectively filter out the high-frequency EMG interference and the thermal noise, while retaining a complete ECG effective signal.

Those of ordinary skill in the art should realize that the above embodiments are only used to illustrate the present disclosure, but not to limit the present disclosure.

As long as they are within the essential spirit of the present disclosure, the above embodiments are appropriately made. Changes and changes fall within the scope of protection of the present disclosure.

What is claimed is:

1. An electrocardiograph signal detecting device comprising:
a first collecting module configured for collecting a first electrocardiogram (ECG) signal;
a filtering module connected to the first collecting module, and configured for filtering the first ECG signal to output a high frequency ECG signal and a low frequency ECG signal;
a second collecting module connected to the filtering module, and configured for collecting the high frequency ECG signal and the low frequency ECG signal; and
a controlling module connected to the second collecting module and configured for performing combination processing the high frequency ECG signal and the low frequency ECG signal, and outputting an effective ECG signal.

2. The electrocardiograph signal detecting device of claim 1, further comprising a displaying module connected to the controlling module and configured for displaying the effective ECG signal.

3. The electrocardiograph signal detecting device of claim 2, wherein the filtering module comprises a power frequency notch filter, a high pass filter and a low pass filter, the power frequency notch filter is electrically connected to the first collecting module and the high pass filter, the low pass filter is electrically connected to the high pass filter and the second collecting module, and the high pass filter is electrically connected to the second collecting module.

4. The electrocardiograph signal detecting device of claim 3, wherein the power frequency notch filter is configured to filter a power frequency noise signal in the first ECG signal and output a second ECG signal.

5. The electrocardiograph signal detecting device of claim 4, wherein the high pass filter is configured to filter a baseline drift signal in the second ECG signal and output the high frequency ECG signal.

6. The electrocardiograph signal detecting device of claim 5, wherein the low pass filter is configured to filter the high frequency noise signal of T-P band in the high frequency ECG signal and output the low frequency ECG signal.

7. The electrocardiograph signal detecting device of claim 1, wherein the controlling module comprises a detecting module, the wave combining module is electrically connected to the detecting module and the second collecting module, the detecting module is configured to detect R peak of the high frequency ECG signal and identify complete QRS complex.

8. The electrocardiograph signal detecting device of claim 7, wherein the controlling module comprises a wave combining module, the wave combining module is configured to perform combination processing the QRS complex and the low frequency ECG signal, and output the ECG effective signal.

9. The electrocardiograph signal detecting device of claim 8, wherein the wave combining module is configured to calculate filter delay and phase difference, and combine waveforms of same frequency band in the QRS complex and the low frequency ECG signal.

10. An electrocardiograph signal detecting method comprising:
collecting a first electrocardiogram (ECG) signal;
filtering the first ECG signal to output a high frequency ECG signal and a low frequency ECG signal;
detecting R peak on the high frequency ECG signal to obtain QRS complex; and
performing combination processing the QRS complex and the low frequency ECG signal, and obtaining an effective ECG signal.

11. The electrocardiograph signal detecting method of claim 10, further comprising:
displaying the effective ECG signal.

12. The electrocardiograph signal detecting method of claim 11, wherein filtering the first ECG signal comprising:
filtering the first ECG signal, and obtaining the high frequency ECG signal; and
filtering the high frequency ECG signal to filter out a high frequency noise signal in T-P band and obtain the low frequency ECG signal.

13. The electrocardiograph signal detecting method of claim 12, wherein obtaining the high frequency ECG signal comprising:
filtering the first ECG signal to filter a power frequency noise signal and obtain a second ECG signal; and
filtering the second ECG signal to filter a baseline drift signal and obtain the high frequency ECG signal.

14. The electrocardiograph signal detecting method of claim 13, wherein performing combination processing the QRS complex and the low frequency ECG signal comprising:
calculating filter delay and phase difference of the QRS complex and the low frequency ECG signal; and
combing waveforms of same frequency band in the QRS complex and the low frequency ECG signal.
